(19) United States
(12) Patent Application Publication
(10) Pub. No.: US 2010/0200000 A1
(43) Pub. Date: Aug. 12, 2010

(54) Penile Cap

(76) Inventor: William Nkoro, Port Coquitlam (CA)

Correspondence Address: William Nkoro, 1434 East 29th Avenue, Vancouver, V5N 2Y5, omitted

(21) Appl. No.: 12/367,522

(22) Filed: Feb. 8, 2009

(51) Int. Cl. A61F 6/04

(52) U.S. Cl. 128/844

(57) ABSTRACT

The Penile Cap is a tubular contraceptive device with a wide open end called the attachment rim, and a narrow closed end called the flexible discharge tube. The penile cap can be made from latex rubber or any other material. The attachment rim consists of a non-toxic glue on its surface for attachment on the penile hole of the male penis thereby providing a tight seal around the penile hole of the male penis. The semen from the male penis is collected in the flexible discharge tube of the penile cap.

### THE PENILE CAP

The Penile Cap is a contraceptive device I designed for men in order to increase sexual stimulation and enjoyment while having sexual intercourse and at the same time serving as a very effective contraceptive device. The Penile Cap is attached to the penile hole and held firmly by a glue. The semen discharge from the male is collected in the discharge tube of the penile cap. The Penile Cap can be made from latex rubber or any other material.

### PROCESS OF MAKING THE PENILE CAP

Collecting the Raw Materials:

Rubber latex is obtained from the milky fluid produced by various tropical plants. Latex is actually an emulsion or dispersion of tiny rubber particles in water, and ingredients added to the latex must be able to attach to the rubber particles during compounding.

Compounding:

Next, chemical additives are mixed to form a paste. This paste is then blended with the liquid latex in a process called compounding.

Storage:

The latex and chemical compound is then unloaded into drums for storage, where it remains for approximately seven days. During this period, vulcanization chemically strengthens the bonds of the rubber. The storage time also allows any air, which might have been trapped in the mixture during compounding, to escape.

Dipping:

The compound is then added to the dipping or penile cap-forming machine. The dipping machine is a long, hooded machine approximately 100 feet (30.5 m) in length. Thick tempered glass rods move along a closed belt between two circular gears. The belt drags the rods, which are called mandrels, through a series of dips into the latex compound. The mandrels rotate to spread the latex evenly. Several coats are required to build the penile cap to its required thickness. Between each dip, the latex is hot air dried.

After the final dipping and drying, the penile caps automatically roll off the mandrels. A machine shapes and trims the ring of latex at the base of each penile cap.

Tumbling:

Next, the penile caps are put in a tumbling machine, where they are coated with talc or another similar powder to prevent the rubber from sticking to itself.

Testing:

After a curing period of several days, the penile caps are sampled by batch and tested for leaks and strength. The first such test is the inflation test, in which the penile cap is filled with air until it bursts. Penile caps are required to stretch beyond 1.5 cubic feet, about the size of a watermelon, before bursting. This test is considered most important because the elasticity of the penile cap keeps it from tearing during inter-course.

In the water-leakage test, the penile cap is filled with 10 ounces (300 ml) of water and inspected for pin-sized holes by rolling it along blotter paper.

Penile caps are also tested electronically. This involves mounting each penile cap on a charged stainless steel mandrel. The mandrel is passed over by a soft, conductive brush. If pin holes are present, a circuit will be established with the mandrel, and the machine will automatically reject the penile cap.

Packaging:

Penile caps that have successfully passed these tests are rolled by a machine. Rolling the penile cap makes it easier to package and use. Spermicide may be applied by a metering pump just before a special organic non-toxic glue or adhesive is applied to the rim of the penile cap.

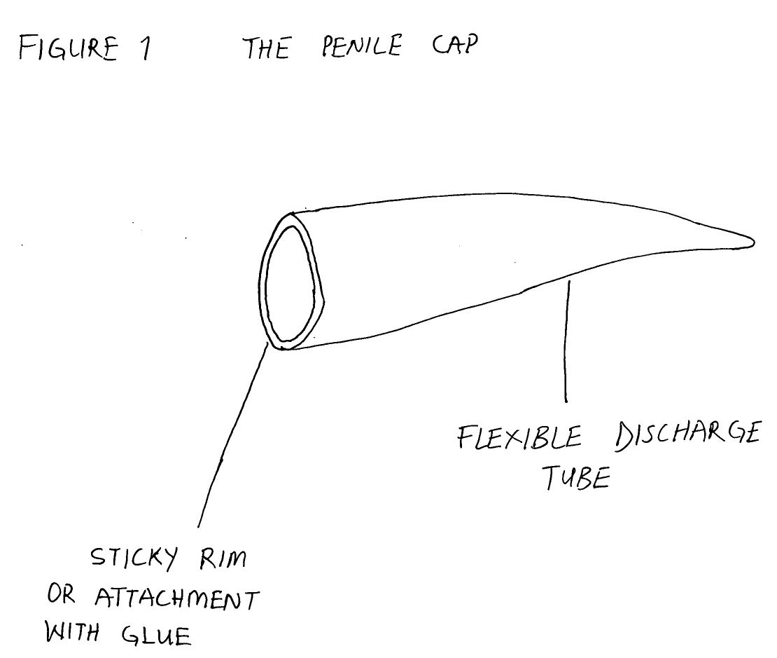

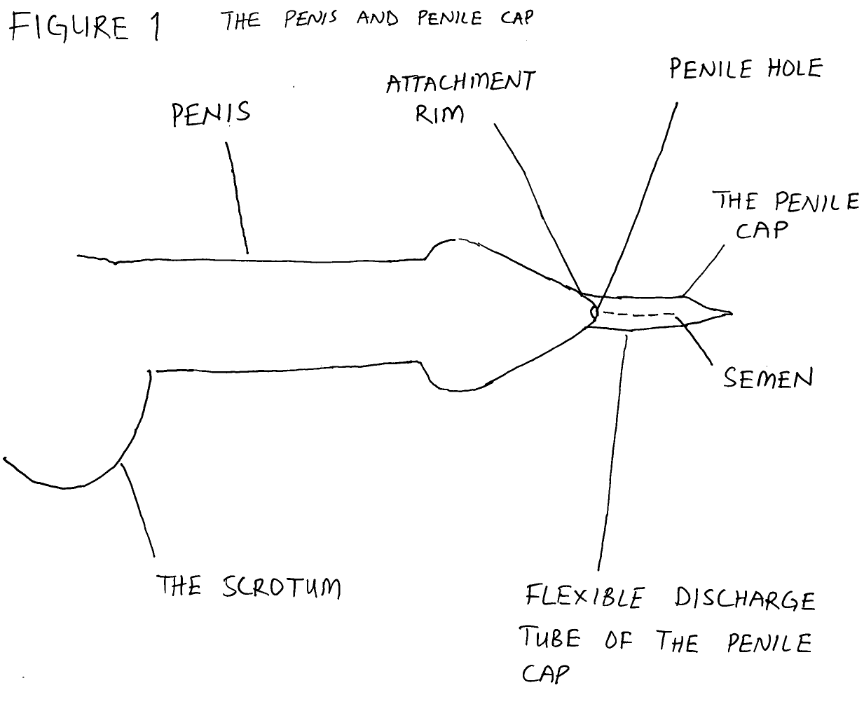

Description of the Drawings: FIG. 1 of diagram I shows the diagram of the penile cap, a tubular device with a narrowly closed end called the flexible discharge tube, and a widely open end called the attachment rim. The attachment rim consists of a non-toxic glue on its surface. This tubular device can be made from latex rubber or any other material.

FIG. 1 of diagram II shows how the penile cap is attached to the tip of the penis to form a tight seal around the penile hole. It also shows the collection of semen from the penis into the flexible discharge tube of the penile cap.

1. The subject matter I regard as my invention in the Penile Cap is the sticky rim that is attached to the penile hole by means of a glue.
